(19) United States
(12) Patent Application Publication
(10) Pub. No.: US 2020/0276186 A1
(43) Pub. Date: Sep. 3, 2020

(54) USE OF MORPHINANS FOR TREATING COCAINE ADDICTION, PRURITIS, AND SEIZURE DISORDERS

(71) Applicant: PHOENIX PHARMALABS, INC., Providence, UT (US)

(72) Inventors: John LAWSON, Hyde Park, UT (US); Lawrence TOLL, Palm City, FL (US)

(21) Appl. No.: 16/765,629

(22) PCT Filed: Nov. 20, 2018
(86) PCT No.: PCT/US2018/061990

§ 371 Date: May 20, 2020

Related U.S. Application Data
(60) Provisional application No. 62/588,681, filed on Nov. 20, 2017.

(51) Int. Cl. A61K 31/485; A61P 25/30

(52) U.S. Cl. CPC A61K 31/485; A61K 9/0053; A61P 25/30

(57) ABSTRACT

The disclosure provides methods of using certain morphinans of Formula (I) that are partial kappa opioid receptor agonists, weak mu opioid receptor agonists, and partial or weak delta opioid receptor agonists in the treatment of kappa opioid receptor-associated diseases and conditions. The disclosure provides methods of treating pruritus, acute seizures, and seizure disorders. The disclosure also provides a method of treating cocaine addiction by administering a particular compound of Formula I, PPL-103, to a cocaine addicted patient.

### CROSS REFERENCE TO RELATED APPLICATION

This application claims priority of U.S. Provisional Appl. No. 62/588,681 file Nov., 20, 2017, which is hereby incorporated by reference in its entirety.

### FIELD OF THE DISCLOSURE

Methods of treating pruritus (itch) and seizure disorders with certain morphinans are provided. Methods of treating cocaine addiction, with a particular morphinan, PPL-103 are also provided. The morphinans useful in these methods are partial kappa agonists.

### BACKGROUND

Pruritus, or itch, is a common condition that is often refractory to existing treatments. Systemic stimulation of kappa-opioid receptors with kappa receptor agonists has been shown to reduce itch in humans and animals.

Seizures are a change in behavior, or spasms or convulsions that arise as a result of a natural chemical imbalance or electrical imbalance in the brain. Such convulsions can be due to a disease or disorder (e.g., epilepsy), age, or the occurrence of a neurological event (e.g., stroke). Epilepsy afflicts approximately 0.3 to 0.5 percent of the world's population. Seizures can be fatal and can also cause progressive memory deterioration. Kappa opioid receptor agonists have been shown to have anticonvulsant and neuroprotective activity. However, because kappa agonists also typically exhibit mu- and delta-opioid receptor activity associated with sedation and addiction potential, they have not been developed for chronic treatment of seizure disorders.

Cocaine produces its psychoactive and addictive effects by causing a buildup of dopamine in the brain. Kappa opioid agonists suppress dopamine release, thereby decreasing the rewarding and euphoric effects of cocaine. Due to the high abuse potential of most opioids cocaine addiction is not currently treated with opioid receptor agonists.

Opioids are the oldest and most prescribed analgesics, primarily as a first-line choice for acute and chronic surgical, cancer, and back pain. Opioids are divided into two primary classes: (a) “mu-active” drugs (e.g., morphine), which are selective for the mu-opioid receptor, and (b) “mixed agonist/antagonist” drugs (e.g., butorphanol, nalbuphine), which typically recognize mu- and kappa-opioid receptors. While opioids are effective in their primary indications, they elicit many limiting side effects, including constipation, respiratory and cardiovascular depression, nausea, urinary retention/diuresis, sedation, dysphoria, tolerance, and/or physical dependence, which seem virtually inseparable from their analgesic effects. Due to such problems, pain patients sometimes take less than the prescribed dosage and/or endure pain rather than suffer from side effects. Such problems also plague physicians, who must monitor patients closely, rotate different drugs to determine the most tolerable drug and dosage, and/or administer extra medicines to counteract side effects.

Morphinans are compounds based on the core chemical structure

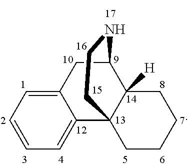

Morphine, a widely used and powerful analgesic, is a common example of a morphinan. Morphine is an opioid with strong agonist effects at the mu opioid receptor. However, the drug has serious side effects that present severe clinical problems, including drug dependence, suppression of respiration, alcohol interaction, and suppression of smooth muscle movement. Other morphinan analogs have weaker agonist activity at the mu opioid receptor and greater affinity for the delta or kappa opioid receptors. Morphinan analogs with reduced mu agonist activity have lower chemical dependency liability and greater safety than morphine and may exhibit other useful biological properties.

### SUMMARY

Applicants have discovered that a class of morphinan compounds is useful for treating pruritus (itch) and seizure disorders. Applicants have also discovered that a particular morphinan, PPL-103 is useful for treating cocaine addiction.

The compounds used in the methods of this disclosure are compounds of Formula (I) and their pharmaceutically acceptable salts.

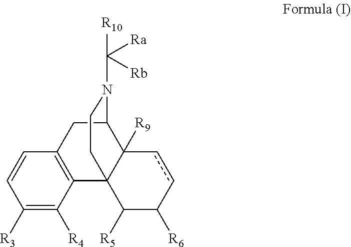

The dashed line between in Formula I represents an optional double bond.

The variables R₃, R₄, R₅, R₆, R₉, R₁₀, Rₐ, and R_{b} have the definitions given below and the definition of each variable is independent of the definition of any of the other variables.

R₃ is hydrogen, halogen, hydroxyl, C₁-C₄alkyl, C₁-C₄alkoxy, or C₁-C₄alkyl ester.

R₄ and R₅ are both hydrogen or R₄ and R₅ are taken together to form a 5 membered ring, optionally containing one O or S ring atom.

R₆ is hydrogen, halogen, hydroxyl, oxo, C₁-C₄alkyl, C₁-C₄alkoxy, or C₁-C₄alkyl ester.

R₉ is hydrogen or methyl.

Rₐ and R_{b} are independently chosen from hydrogen, halogen, and C₁-C₄alkyl. It is preferred that Rₐ and R_{b} are not both hydrogen.

R₁₀ is C₁-C₆alkyl, C₂-C₆alkenyl, or (C₃-C₆cycloalkyl)C₀-C₂alkyl.

Methods of treating pruritus or seizure disorders comprising administering an effective amount of a compound or salt of Formula (I) or any subformula of Formula (I) to a patient in need of such treatment are provided. Methods of treating cocaine addiction comprising administering PPL-103, a compound of Formula (I) in which R₃ and R₆ are both hydroxyl, R₉ is hydrogen, Rₐ is hydrogen, R_{b} is methyl and R₁₀is cyclopropyl are provided herein. In all instances in which Formula (I) is mentioned hereing, PPL-103 is a particular embodiment.

### BRIEF DESCRIPTION OF THE DRAWINGS

FIG. 1. Effect of PPL-103 of cocaine self-administration (FR-1, 20 seconds time on). FIG. 1 shows the number of times rats self-administered cocaine during a 30 minute interval when dosed with vehicle or PPL-103, 1 mg/kg). FIG. 1 shows a small but significant reduction in cocaine self administration.

FIG. 2. Effect of PPL-103 on cocaine priming (10 mg/kg) reinstatement of cocaine seeking. FIG. 2 shows that PPL-103 is very good at blocking relapse.

### DETAILED DESCRIPTION

### Terminology

Before describing the invention in detail, it will be helpful to have these definitions of terms used in the claims and elsewhere in the specification. Compounds are described using standard nomenclature.

Unless otherwise indicated, the disclosure is not limited to specific procedures, starting materials, or the like, as such may vary. It is also to be understood that the terminology used herein is for the purpose of describing particular embodiments only and is not intended to be limiting. Unless clearly contraindicated by the context each compound name includes the free acid or free base form of the compound as well as hydrates and pharmaceutically acceptable salts of the compound.

The terms “a” and “an” do not denote a limitation of quantity, but rather denote the presence of at least one of the referenced item. The term “or” means “and/or”. The terms “comprising”, “having”, “including”, and “containing” are to be construed as open-ended terms (i.e., meaning “including, but not limited to”). The open ended term “comprising” encompasses the terms “consisting of” and “consisting essentially of.”

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable. All methods described herein can be performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

In describing and claiming the present invention, the following terminology will be used in accordance with the definitions set out below.

The phrases “for example,” “for instance,” “such as,” or “including” are meant to introduce examples that further clarify more general subject matter. These examples are provided only as an aid for understanding the disclosure, and are not meant to be limiting in any fashion.

The terms “optional” and “optionally,” mean that the subsequently described circumstance may or may not occur, so that the description includes instances where the circumstance occurs and instances where it does not. For example, the phrase “optionally substituted” means that a non-hydrogen substituent may be present on a given atom, and, thus, the description includes structures wherein a non-hydrogen substituent is present and structures wherein a non-hydrogen substituent is not present.

The term “independently selected from” is used herein to indicate that the recited elements, e.g., R groups or the like, can be identical or different.

“Alkyl” is a branched or unbranched saturated hydrocarbon group generally containing 1 to about 8 carbon atoms, such as methyl, ethyl, n-propyl, isopropyl, n-butyl, isobutyl, t-butyl, octyl, decyl, or the specified number of carbon atoms. Other embodiments include alkyl groups having from 1 to 6 carbon atoms, 1 to 4 carbon atoms or from 1 to 2 carbon atoms, e.g. C₁-C₆alkyl, C₁-C₄alkyl, and C₁-C₂alkyl. When C₀-Cₙ alkyl is used herein in conjunction with another group, for example, (cycloalkyl)C₀-C₄ alkyl, the indicated group, in this case cycloalkyl, is either directly bound by a single covalent bond (C₀), or attached by an alkyl chain having the specified number of carbon atoms, in this case from 1 to about 4 carbon atoms.

“Alkyl ester is a an alkyl group as described above bound to the position it substitutes via an ester group, either —(C═O)O— or —O(C═O)—.

“Alkenyl” is a straight or branched hydrocarbon chain comprising one or more unsaturated carbon-carbon double bonds, which may occur in any stable point along the chain. Alkenyl groups described herein have the indicated number of carbon atoms. E.g. C₂-C₆alkenyl indicates an alkenyl group of from 2 to about 6 carbon atoms. When no number of carbon atoms is indicated, alkenyl groups described herein typically have from 2 to about 8 carbon atoms, though lower alkenyl groups, having 6 or fewer carbon atoms, are preferred. Examples of alkenyl groups include ethenyl, propenyl, and butenyl groups.˜

“Alkoxy” is an alkyl group as defined above with the indicated number of carbon atoms attached through an oxygen bridge (—O—). Examples of alkoxy include, but are not limited to, methoxy, ethoxy, n-propoxy, i-propoxy, n-butoxy, 2-butoxy, t-butoxy, n-pentoxy, 2-pentoxy, 3-pentoxy, isopentoxy, neopentoxy, n-hexoxy, 2-hexoxy, 3-hexoxy, and 3-methylpentoxy. “Halo” and “halogen” mean a chloro, bromo, fluoro or iodo substituent.

“Oxo,” is a keto group (C═O). An oxo group that is a substituent of a nonaromatic carbon atom results in a conversion of —CH₂— to —C(═O)—. An oxo group that is a substituent of an aromatic carbon atom results in a conversion of —CH — to —C(═O)— and a loss of aromaticity.

The term “enantioenriched” is used to indicate that, where a compound may exist as two or more enantiomers, one of the enantiomers is present in excess of the other(s). For example, where two enantiomers of a compound are possible, an enantioenriched sample may include greater than 50%, greater than 60%, greater than 70%, greater than 75%, greater than 80%, greater than 85%, greater than 90%, greater than 95%, or greater than 99% of one of the enantiomers. A process is “enantioenriching” or “enantioselective” when the process favors production of one enantiomer over production of another enantiomer. Similarly, the term “diastereomerically enriched” is used to indicate that, where a compound may exist as two or more diastereomers, one of the diastereomers is present in excess of the other(s). For example, where two diastereomers of a compound are possible, a diastereomerically enriched sample may include greater than 50%, greater than 60%, greater than 70%, greater than 75%, greater than 80%, greater than 85%, greater than 90%, greater than 95%, or greater than 99% of one of the diastereomers. A process is “diastereomerically enriching” or “diastereoselective” when the process favors production of one diastereomer over production of another diaseteomer.

Unless otherwise specified, reference to an atom is meant to include isotopes of that atom. For example, reference to H is meant to include ¹H, ²H (i.e., D) and ³H (i.e., T), and reference to C is meant to include ¹²C and all isotopes of carbon (such as ¹³C).

“Pharmaceutical compositions” are compositions comprising at least one active agent, such as a compound or salt of Formula (I), and at least one other substance, such as a carrier, excipient, or diluent. Pharmaceutical compositions meet the U.S. FDA's GMP (good manufacturing practice) standards for human or non-human drugs.

“Pharmaceutically acceptable salts” includes derivatives of the disclosed compounds in which the parent compound is modified by making inorganic and organic, non-toxic, acid or base addition salts thereof. The salts of the present compounds can be synthesized from a parent compound that contains a basic or acidic moiety by conventional chemical methods. Generally, such salts can be prepared by reacting free acid forms of these compounds with a stoichiometric amount of the appropriate base (such as Na, Ca, Mg, or K hydroxide, carbonate, bicarbonate, or the like), or by reacting free base forms of these compounds with a stoichiometric amount of the appropriate acid. Such reactions are typically carried out in water or in an organic solvent, or in a mixture of the two. Generally, non-aqueous media like ether, ethyl acetate, ethanol, isopropanol, or acetonitrile are preferred, where practicable. Salts of the present compounds further include solvates of the compounds and of the compound salts.

Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts and the quaternary ammonium salts of the parent compound formed, for example, from non-toxic inorganic or organic acids. For example, conventional non-toxic acid salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, mesylic, esylic, besylic, sulfanilic, 2-acetoxybenzoic, fumaric, toluenesulfonic, methanesulfonic, ethane disulfonic, oxalic, isethionic, HOOC—(CH₂)ₙ—COOH where n is 0-4, and the like.

The term “carrier” applied to pharmaceutical compositions of the invention refers to a diluent, excipient, or vehicle with which an active compound is provided.

“Providing” means giving, administering, selling, distributing, transferring (for profit or not), manufacturing, compounding, or dispensing.

“Providing a compound of Formula (I) with at least one additional active agent” means the compound of Formula (I) is a first active agent and it is provided with the additional active agent(s) either simultaneously in a single dosage form, concomitantly in separate dosage forms, or provided in separate dosage forms for administration separated by some amount of time that is within the time in which both the compound of Formula (I) and the at least one additional active agent are within the blood stream of a patient. The compound of Formula (I) and the additional active agent need not be prescribed for a patient by the same medical care worker. The additional active agent or agents need not require a prescription. Administration of the compound of Formula (I) or the at least one additional active agent can occur via any appropriate route, for example, oral tablets, oral capsules, oral liquids, inhalation, injection, intravenous administration, suppositories or topical contact. In certain embodiments the compound of Formula (I) will be administered as a long acting injection, i.e. a depot formulation.

“Treatment,” as used herein includes providing a compound of Formula (I) to a patient a patient having pruritus, a seizure disorder, or cocaine addiction. The term “treatment” or “treating” refers to inhibiting or slowing the progression of a disease or disorder, e.g., epilepsy, stabilization of a discernible symptom, such as seizures, delaying the onset of seizures, or, reduction in the severity of symptoms, such as reduction in severity or intensity of itch. In certain embodiments treatment may be prophylactic, meaning that a compound of Formula (I) delays or prevents the onset of symptoms of a disorder. A compound of Formula (I) can be administered as a preventative measure to a subject having a genetic or non-genetic predisposition to epilepsy or at risk of developing seizures as a result of another medical event. For example, patients who have suffered a stroke are at risk of developing seizures. In these instances, the compound of Formula (I) can be administered after the stroke as a preventative measure against seizures.

A “therapeutically effective amount” of a compound of Formula (I) means an amount effective, when administered to a patient, to provide a therapeutic benefit such as an amelioration of at least on symptom of the disorder, decrease the frequency or severity of symptoms, or effect a change in a clinical marker for a disease or disorder. For example a therapeutically effective amount of a compound of Formula (I) is an amount effective to decrease pruritus symptoms or reduce the frequency or severity of seizures.

### Chemical Description

In Formula (I) the variables, e.g. R₃, R₆, R₉, R₁₀, Rₐ, and R_{b} can have the definitions listed in the “Summary” section or any of the definitions listed in this section. The methods of treatment of this disclosure include using compounds of Formula (I) having any combination of variable definitions set forth in the Summary section or in this section so long as a stable compound results.

Compounds of Formula (Ia)-Formula (Ij), which are subformulae of Formula (I), may be used in the methods of this disclosure.

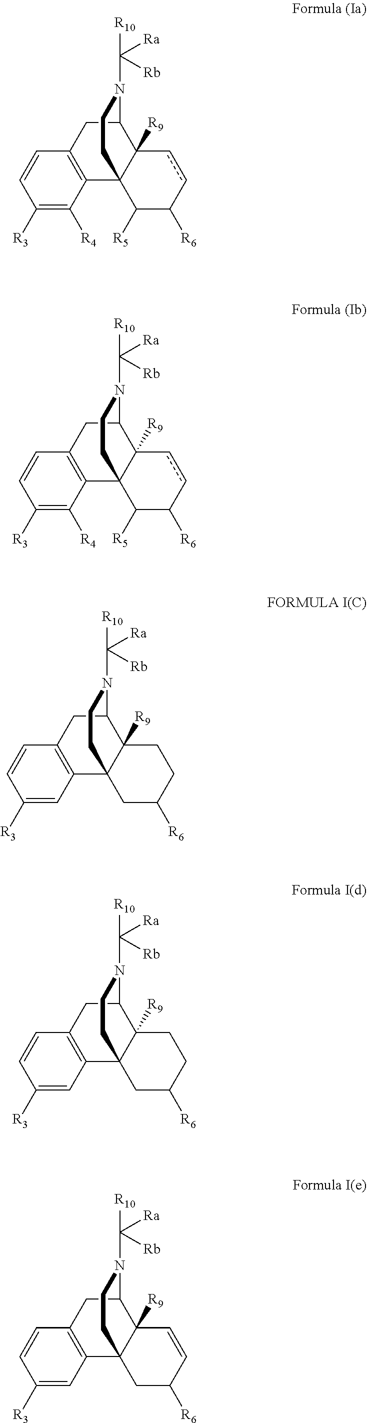

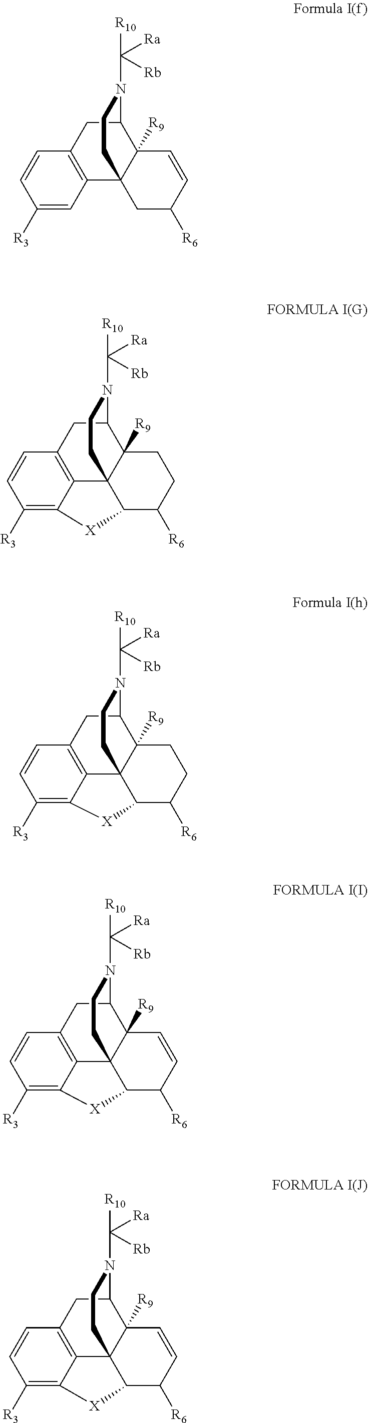

In one embodiment R₃ is hydrogen or hydroxyl, and R₆ is hydrogen, hydroxyl, or oxo, R₉ is hydrogen or methyl, and X if present is O.

Ra and Rb are independently hydrogen, halogen, methyl, or ethyl.

This disclosure also provides a compound or pharmaceutically acceptable salt thereof of Formula (IIIa and IIIb) (which are subformulae of Formula (I)).

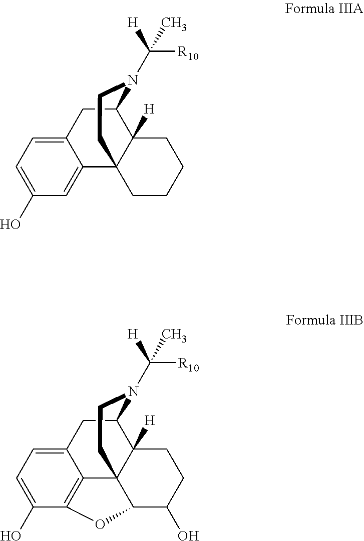

In Formula IIIa and IIIb, R₁₀ includes cyclopropyl, ethyl, propyl, or vinyl.

Also included are the following specific embodiments, compounds of Formula IVa-Formula IVh. Formula IVa is PPL-103.

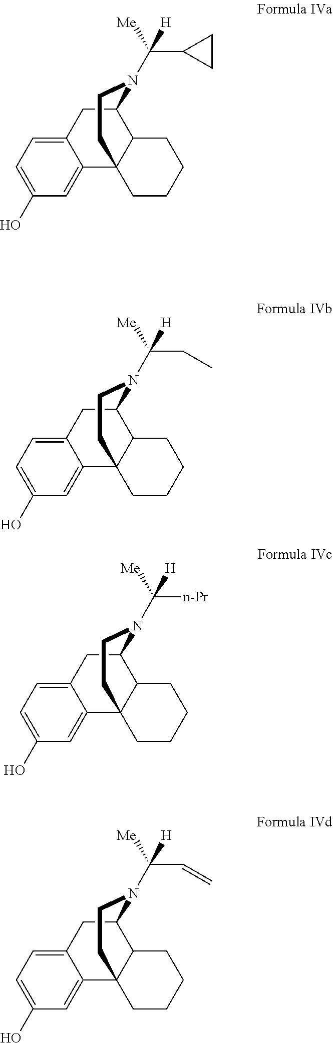

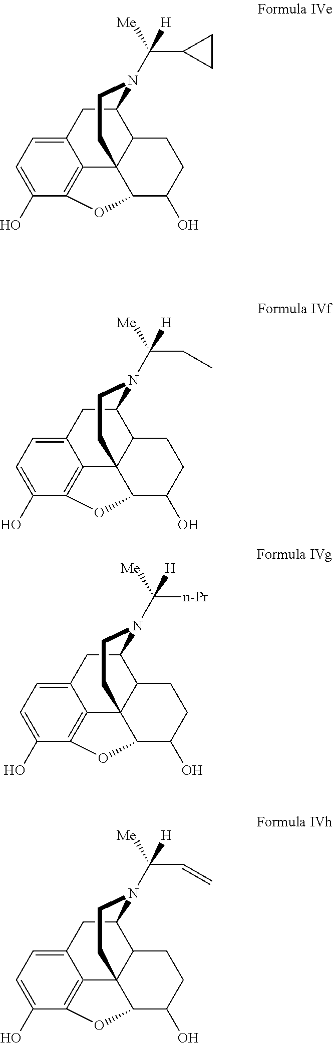

### Pharmaceutical Preparations

Compounds of Formula (I) can be administered as the neat chemical, but are preferably administered as a pharmaceutical composition. Accordingly, the disclosure provides pharmaceutical compositions comprising a compound or pharmaceutically acceptable salt of the Formula (I), together with at least one pharmaceutically acceptable carrier. The pharmaceutical composition may contain a compound or salt of Formula (I) as the only active agent, or may contain one or more additional active agents.

Compounds of Formula (I) may be administered orally, topically, parenterally, by inhalation or spray, sublingually, transdermally, intravenously, intrathecally, via buccal administration, rectally, as an ophthalmic solution, or by other means, in dosage unit formulations containing conventional pharmaceutically acceptable carriers. The pharmaceutical composition may be formulated as any pharmaceutically useful form, e.g., as an aerosol, a cream, a gel, a pill, a capsule, a tablet, a syrup, a transdermal patch, or an ophthalmic solution. Some dosage forms, such as tablets and capsules, are subdivided into suitably sized unit doses containing appropriate quantities of the active components, e.g., an effective amount to achieve the desired purpose.

Carriers include excipients and diluents and must be of sufficiently high purity and sufficiently low toxicity to render them suitable for administration to the patient being treated. The carrier can be inert or it can possess pharmaceutical benefits of its own. The amount of carrier employed in conjunction with the compound is sufficient to provide a practical quantity of material for administration per unit dose of the compound.

Classes of carriers include, but are not limited to binders, buffering agents, coloring agents, diluents, disintegrants, emulsifiers, flavorants, glidents, lubricants, preservatives, stabilizers, surfactants, tableting agents, and wetting agents. Some carriers may be listed in more than one class, for example vegetable oil may be used as a lubricant in some formulations and a diluent in others. Exemplary pharmaceutically acceptable carriers include sugars, starches, celluloses, powdered tragacanth, malt, gelatin; talc, and vegetable oils. Optional active agents may be included in a pharmaceutical composition, which do not substantially interfere with the activity of the compound of the present invention.

The pharmaceutical compositions can be formulated for oral administration. These compositions contain between 0.1 and 99 weight % (wt. %) of a compound of Formula (I) and usually at least about 5 wt. % of a compound of Formula (I). Some embodiments contain from about 25 wt. % to about 50 wt. % or from about 5 wt. % to about 75 wt. % of the compound of Formula (I).

### Methods of Use

This disclosure includes methods of treating pruritus, seizure disorders, and cocaine addiction comprising administering a therapeutically effective amount of a compound of Formula (I) to a patient in need of such treatment.

Pruritus a condition of localized or general itching, that is a symptom of a variety of diseases and disorders. Pruritus usually occurs in the skin, but sometime occurs in non-cutaneous tissues such as mucus membranes. Pruritus may be due to localized skin disorders caused by allergic reactions, dermatitis (including contact dermatitis, atopic dermatitis, seborrheic dermatitis), insect bites, urticaria, asteatosis, senile pruritus cutaneous, photosensitive dermatosis, prurigo, herpes, impetigo, eczema, tinea, lichen, psoriasis, scabies, acne vulgaris, dermatoses caused by bacterial or fungal infections, parasitic infections, and hemorrhoids. Pruritus may also be due to systemic disease and conditions such as hypothyroidism, thyrotoxicosis, kidney failure (uraemic pruritus), liver disease (cholestatic pruritus), prurigo nodularis, mucocandiasis in diabetes mellitus, pregnancy (pruritic urticarial papules and plaques of pregnancy), malignant tumors, and Hodgkin's disease.

The disclosure includes methods of treating acute seizures and seizure disorders. A “seizure” as used herein is a change in behavior, or spasms or convulsions that arise in a subject as a result of a natural chemical imbalance or electrical imbalance in the brain. Such convulsions can be due to a disease or disorder (e.g., epilepsy), age, or the occurrence of a neurological event (e.g., stroke). The term “seizure” also refers to seizures that are chemically induced, for example those brought on by intake, uptake, or ingestion of chemicals such as organophosphates.

Seizures are classified epileptic and non-epileptic, The disclosure includes methods of treating non-epileptic seizures including provoked seizures due to injury, stroke, brain tumors, drug exposure, fever, febrile seizures, heart attack, sleep deprivation, infection or fever, low oxygen levels, or very low blood sugar.

Epileptic seizures are caused by excessive and/or hypersynchronous abnormal activity of neurons in the cerebral cortex, such as disturbances in the excitatory-inhibitory equilibrium of neuronal activity. The neuromechanism responsible for seizures includes the amygdala, the hippocampus, the hypothalamus, the parolfactory cortex, the frontal and temporal lobes, and the involvement of the substantia nigra.

Epileptic seizures can be classified as partial and generalized seizures. Partial seizures originate in specific brain regions and remain localized commonly in the temporal lobes (containing the hippocampus), whereas generalized seizures occur in the entire forebrain and affect both cerebral hemispheres from the beginning of the seizure. Generalized seizures produce a loss of consciousness and may be prolonged, Generalized seizures include Grand mal, absence, myoclonic, tonic, clonic, and atonic seizures.

In partial seizures the onset of the electrical disturbance is limited to a specific area of one cerebral hemisphere. Partial seizures are classified as simple partial seizures (in which consciousness is retained); and complex partial seizures (in which consciousness is impaired or lost) Partial seizures may spread to the other cerebral hemisphere causing a generalized seizure. Partial seizures that become generalized are termed “partial seizures secondarily generalized. Partial seizures are the most common type of epileptic seizure, Virtually any movement, sensory, or emotional symptom, including complex visual or auditory hallucination, can occur as part of a partial seizure. There are two types of partial seizure, simple partial seizures and complex partial seizures. Complex partial seizures generally affect a larger area of the brain than simple partial seizures and they affect consciousness. Complex partial seizures can affect any area of the brain, but often affect one or both of the brain's two temporal lobes. Because of this, complex partial seizures are sometimes called “temporal lobe epilepsy.”

Seizure disorders include epilepsy. Methods of treating epilepsy provided by this disclosure include methods of treating West syndrome, Doose syndrome, benign rolandic epilepsy, Rasmussen's syndrome, Lennox-Gastaut syndrome, Landau-Kleffner syndrome, Sturge-Weber syndrome, juvenile myoclonic epilepsy, frontal lobe epilepsy, temporal lobe epilepsy, parietal lobe epilepsy, and occipital lobe epilepsy. Method of treating epilepsy provided by this disclosure include treating epilepsies in which the seizures are refractory, occurring for prolonged more than 30 minutes without relief. Refractory epilepsies that can be treated with compounds of Formula (I) include status epilepticus, refractory status epilepticus, super refractory status epilepticus, or PCDH19 female pediatric epilepsy. Refractory epilepsies generally require treatment in a hospital setting and intravenous administration of anti-seizure medications, such as a compound of Formula (I). Methods of treating refractory epilepsy include methods in which the patient is in an induced coma.

Methods of treatment of this disclosure include administering a therapeutically effective amount of a compound of Formula (I) to a patient actively having a seizure as well as administering an therapeutically effective amount of a compound of Formula (I) to patient known to suffer from seizures or to be at risk for seizures and thereby reducing the likelihood, frequency or severity of seizures in the patient.

Compounds and salts of Formula (I), particularly Compound IVa (PPL-103), are also useful for treatment of cocaine addiction. Without wishing to be bound to any particular theory it is believe that the kappa agonist activity of compounds of Formula (I) acts to suppress dopamine release, thereby decreasing the rewarding and euphoric effects of cocaine. In other words, the opioid system is used to manipulate and downregulate the dopaminergic system, likely by elevating the levels of certain opioid peptides, including endorphins and enkephalins that regulated dopamine synthesis and release. While most opioid agonists cannot be used to treat cocaine addiction due to their own very high potential for addiction and abuse, applicants have surprisingly found that Compound IVa (PPL-103) has a opioid receptor profile that permits down regulation of the dopamine response without the addictive potential associated with most opioid agonists.

In one embodiment a cocaine addicted patient is provided a daily oral dose of a compound of Formula (I). The cocaine content of the addicted patient's urine may be analyzed to determine treatment efficacy, where decreased frequency of urine samples positive for cocaine indicates effective treatment, though preferably an effectively treated patient will consistently have urine samples negative for cocaine. Effective treatment of cocaine addicted patients with a compound of Formula (I) also comprises administering an amount of a compound of Formula (I) sufficient to suppress cumulative withdrawal symptoms of cocaine.

In certain embodiments the compound of Formula (I) is a partial agonist at each of the mu-, kappa-, and delta-opioid receptors and has a high or medium affinity toward each of the mu-, kappa-, and delta-opioid receptors. Compounds of Formula (I) typically exhibit higher affinity for the kappa-opioid receptor than for the mu- or delta-opioid receptors. In certain embodiments the compound of Formula (I) has a binding affinity (Ki) of less than 10.0 nM at each of the mu-, kappa-, and delta-opioid receptors and/or and EC₅₀ value of less than 30 nM at each of the mu-, kappa-, and delta-opioid receptors. In certain embodiment the compound of Formula I is administered as a single diasteromer or as a diasteromerically encriched mixture of diastereomers. Certain compounds of Formula (I) exhibit increased delta opioid receptor affinity. Such compounds may suppress drug tolerance, avert the conditioned rewarding affect associated with many opioids, and block physical dependence on opioids. Certain compounds of Formula (I) exhibit a delta opioid receptor affinity that is not more than 20 fold less, not more than 10 fold less, not more than 5 fold less than the compound's mu opioid receptor affinity. Certain compounds of Formula (I) exhibit kappa agonist activity that is 2 fold greater than, 5 fold greater than or 10 fold greater than the activity of the compound and the mu- or delta-opioid receptor.

Methods of treatment include providing certain dosage amounts of a compound of Formula (I) to a patient. Dosage levels of Formula (I) of from about 0.01 mg to about 140 mg per kilogram of body weight per day are useful in the treatment of the above-indicated conditions (about 0.5 mg to about 7 g per patient per day). In certain embodiments 1 mg to 1000 mg, 1 mg to 500 mg, 1 mg to 100 mg, 1mg to 10 mg, 25 mg to 500 mg, or 25 mg to 200 mg of a compound of Formula (I) are provided daily to a patient.

Frequency of dosage may also vary depending on the compound used and the particular disease treated. However, for treatment of most painful disorders, a dosage regimen of 4 times daily or less is preferred, and a dosage regimen of 1 or 2 times daily is particularly preferred. For treatment of opioid addition a dosage regimen of 1 times daily or less is particularly preferred.

It will be understood, however, that the specific dose level for any particular patient will depend upon a variety of factors including the activity of the specific compound employed, the age, body weight, general health, sex, diet, time of administration, route of administration, and rate of excretion, drug combination and the severity of the particular disorder for the patient undergoing therapy.

### Combination Therapy

Compound of Formula (I) may be used alone or in combination with another active agent.

Suitable does for a compound of Formula (I) when used in combination with a second active agent are generally as described above. Doses and methods of administration of other therapeutic agents can be found, for example, in the manufacturer's instructions in the Physician's Desk Reference. In certain embodiments, the combination administration of a compound of Formula (I) with the second active agent results in a reduction of the dosage of the second active agent required to produce a therapeutic effect (i.e., a decrease in the minimum therapeutically effective amount). Thus, preferably, the dosage of second active agent in a combination or combination treatment method is less than the maximum dose advised by the manufacturer for administration of the second active agent without combination administration of a compound of Formula (I). In certain embodiment this dosage is less than ¾, less than ½, less than ¼, or even less than 10% of the maximum dose advised by the manufacturer for the second active agent when administered without combination administration of a compound of Formula (I).

When treating pruritus a compound of Formula (I) can act as a first active agent in a combination with one or more additional anti-pruritus active agents. The anti-pruritus additional active agent can be menthol, capsaicin, topical salicyclic acid, a topical cannabinoid, topical emollient, a topical immunomodulator, a topical steroid, an antihistamine, a topical anesthetic, an SNRI, an SSRI, a 5HT3 receptor antagonist, a neuroleptic.

Topical emollients include ammonium lactate. Topical immunomodulators include tacrolimus and pimecrolimus Topical steroids include hydrocortisone. Oral steroids include prednisone. Antihistamines include hydroxyzine, diphenhydramine, and cyproheptadine. Topical anesthetics include lidocaine, benzocaine, and pramoxine. SNRI's include mirtazpine for treating pruritus associated with cancer. SSRIs useful for treating pruritus include paroxetine and fluvoxamine. 5HT3 receptor antagonists include ondansetron. Neuroleptics useful for treating pruritus include gabapentin and pregabalin.

When treating seizures or seizure disorders a compound of Formula (I) may be administered to a patient in combination with an additional active agent. The addictive active agent can be an anti-seizure agent or another type of CNS active agent, such as a sedative.

The disclosure includes embodiments in which the compound of Formula (I) is administered in combination with an additional active agent selected from an anti-convulsant (e.g. a Anticonvulsants include GABA_{A} receptor modulators, sodium channel blockers, GAT-1 GABA transporter modulators, GABA transaminase modulators, voltage-gated calcium channel blockers, and peroxisome proliferator-activated alpha modulators), a neurosteroid, and anesthetic.

The disclosure includes embodiments in which the patient is given an anesthetic or sedative in combination with a compound of Formula (I). When treating refractory seizures the anesthetic or sedative may be administered at a concentration sufficient to cause the patient to lose consciousness, such as a concentration sufficient to medically induce coma or a concentration effective to induce general anesthesia. Or the anesthetic or sedative may be given at a lower dose effective for sedation, but not sufficient to induce a loss of consciousness.

Anaesthetics that may be used in combination with a compound of Formula (I) to treat seizures include inhalational anesthetics and intravenous anesthetics which include barbiturate and non-barbiturate anesthetics.

Inhalational anesthetics include desflurane, enflurane, ethyl chloride, halothane, isoflurane, methoxyflurane, sevoflurane, and trichloroethylene.

Intravenous, non-barbiturate anesthetics include atracurium, cisatracurium, etodimidate, ketamine, propofol, and rocuronium.

Barbiturates include amobarbital, methohexital, pentobarbital, phenobarbital, secobarbital, thiamylal, and thiopental.

Benzodiazepines are used both as anticonvulsants and anesthetics. Benzodiazepines useful as anaesthetics include diazepam, flunitrazepam, lorazepam, and midazolam.

Anticonvulsants that may be used in combination with compound of Formula (I) to treat seizuresor seizure disorders include aldehydes, such as paraldehyde; aromatic allylic alcohols. such as stiripentol; barbiturates, including those listed above, as well as methylphenobarbitai and barbexaclone; benzodiazepines include alprazolam, bretazenil, bromazepam, brotizolam, chioridazepoxide, cinolazepam, clonazepam, chorazepate, clopazam, clotiazepam, cloxazolam, delorazepam, diazepam, estazolam, etizolam, ethyl loflazepate, flunitrazepam, flurazepam, flutoprazepam, halazepam, ketazolam, loprazolam, lorazepam, lormetazepam, medazepam, midazolam, nimetazepam, nitrazeparn, nordazeparn, oxazepam, phenenazepam, pinazepam, prazepam, premazepam, pyrazolarn, quazepam, temazepam, tatrazepam, and triazolam; bromides, such as potassium bromide; carboxamides, such carbamazepine, oxcarbazepine, and eslicarbazepine acetate; fatty acids, such as valproic acid, sodium valproate and divalproex sodium, fructose derivatives, such as topiramate; GABA analogs such as gabapentin and pregabalin, hydantoins, such as ethotoin, phenytoin, mephenytoin, and fosphenytoin; other neurosteroids, such as allopregnanolone, oxasolidinediones, such as paramethadione, trimethadione, and ethadione, propionates such as beclamide; pyrimidinediones such as primidone, pyrrolidines such as brivaracetam, levetiracetam, and seletracetarn, succinimides, such as ethosuximide, pensuximide, and mesuximide; sulfonamides such as acetazoloamide, sultiame, methazolamide, and zonisamide; triazines such as lamotrigine, ureas such as pheneturide and phenacemide; NMDA antagonists, such as felbamate, and valproylamides such as valpromide and valnoctamide; and perampanel.

Compounds that may be used in combination with a compound of Formula (I) include vitamins as cocaine addicts frequency suffer from nutritional deficiencies. A compound of Formula (I) may be administered together with pantothenic acid (B5), pyridoxal phosphate (B6), magnesium, calcium, or zinc. Vitamin B6 is a co-factor in the synthesis of dopamine, serotonin and GABA. It is particularly desirable to administer a compound of Formula (I) together with vitamin B6. The compound of Formula (I) may also be combined with a GABA precursor, dopamine precursor, or serotonin precursor. Additional compounds that may be provided together with a compound of Formula (I) to treat cocaine addiction include the dopamine precursors L-Phe, D-dopa, and L-Tyr, serotonin precursors 5-hydroxytryptophan and L-Trp and GABA precursors L-Gln, L, glutamic acid and L-glutamate.

Methods of use include providing a compound of Formula (I) as a packaged composition. Such methods include providing a compound of Formula (I) in a container together with instructions for using the compound to treat pruritus, seizures or a seizure disorder, or cocaine addiction are included. The packaged composition may include one or more additional active agents.

The disclosure provides isotopically and radiolabeled compounds of Formula (I). A compound of Formula (I) may be labeled using any of a variety of well-known techniques (e.g., radiolabeled with a radionuclide such as tritium, as described herein), and incubated with a sample for a suitable incubation time (e.g., determined by first assaying a time course of binding).

### EXAMPLES

### Example 1. Decreased Itching of Topically Treatded Uticaria

Itching intensity in a human patient suffering from uticaria is evaluated prior to treatment. The patient ranks itching intensity as Severe (+++), Moderate (++), Mild (+), or absent (−). A compound of Formula (I) is dissolved in physiological saline, to give an aqueous solution with a concentration of 10-100 μg/ml. This aqueous solution is topically applied to three erupted lesions of urticaria, on the leg or arm of the human patient dose of 0.05 to 50 μg/cm². Itching intensity is evaluated immediately after treatment and at hourly time points for several hours following treatment. For certain compounds of Formula (I) itching whose intensity was evaluated as Severe (+++) or Moderate (++) before treatment is completely eradicated inutes after the treatment (ranked as “−”). The absence of itching continues for several hours after treatment.

### Example 2.Topical Treatment of Dermatitis

An aqueous solution (10-100 μg/ml) of a compound of Formula (I) is applied topically to the skin lesions on the arms or legs of a patient with atopic dermatitis which had caused severe itching (ranked as “+++” by the patient prior to treatment). The drug solution is applied on five spots with a volume of about 50 μl per 10 cm², which was equivalent to a topical dose of 0.05 to 50μg/cm². As a control, indomethacin cream (concentration of 7.5 mg/g) applied at a dose of 75 μg/cm² in the same manner. Itching intensity is evaluated immediately after treatment, 5 minutes after treatment and at hourly time points for several hours following treatment. For certain compounds of Formula (I) itching significantly reduced or eliminated itch at 5 minutes after treatment. Itch relief typically continues for several hours following treatment. In contrast indomethecin reduces but fails to eliminate itch.

### Example 3. Reduction of Nor-BNI Induced Scratching

Reduction of scratching behavior induced by subcutaneous administration of the potent and selective K receptor antagonist, non-binaltorphimine (nor-BNI), into the rostral back of ddY mice, by a compound of Formula (I) is evaluated by this assay. As a negative control, a buffer adjusted to pH 4-6 was administered subcutaneously into the rostral back of mice instead of nor-BNI. The administration of buffer did not elicit any scratching behavior in the mouse.

A compound of Formula (I) is dissolved in physiological saline. This aqueous solution is administered intraperitoneally to mice at a dose of 1 to 10 mg/kg. 30 minutes after administration of the Compound of Formula (I), a 0.1% solution of nor-BNI is administered subcutaneously into the rostral back skin of the mouse at a volume of 0.1 ml/10 g (body weight) to induce scratching. Following the nor-BNI administration, the number of scratching behaviors is counted over 60 minutes.

The following positive control is performed in a separate testing session. Chlorophenylamine, an anti-histamine agent is dissolved in physiological saline. This solution is administered intraperitoneally to mice at a dose of 0.3 to 3.0 mg/kg. Thirty minutes after the administration, nor-BNI was administered and the number of scratching behaviors is counted as previously described.

In a third session, to provide an additional negative control, mice were injected with saline, followed by nor-BNI administration. The number of scratching behaviors in this group was counted over 60 minutes.

The experiments and controls described above were each performed on groups of 10 mice. Certain compounds of Formula (I) are found to reduce scratching behavior relative to the negative control. Certain preferred compounds of Formula (I) reduce scratching behavior more than the positive control, chlorophenylamine.

### Example 4. Evaluation of Anti-Seizure Effects of Compounds in Maximal Electroshock Model

The maximal electroshock (MES) is used to evaluate the efficacy of therapeutic agents against grand mal seizures. Mice, 18-25g, are used in the experiment. The animals are injected with of 0.1-0.3 mg/kg compound of Formula (I) in 0.9% saline prior to electroshock. Maximal seizures are induced by the application of electrical current to the brain via corneal electrodes. The stimulus parameters for mice are 50 mA in a pulse of 60 Hz for 200 ms. and spasm inhibition was recorded as a measure of anticonvulsant activity. Mice in the control group injected with 0.9% saline without drug and are then electroshocked. 4 mice were used for each control and experimental group. Mice are evaluated for seizure activity immediately following electroshock and at 30 minutes post electroshock.

### Example 5. Evaluation of Anti-Seizure Effects of Compounds in the Subcutaneous Metrazole Seizure Model

The Subcutaneous Metrazole (SCMET) model is used to evaluate compound efficacy against petite mal seizures. A Metrazole dose of 85 mg/kg was administered subcutaneously to induce seizures. Mice, 18-25 g, are then injected with 0.1-0.3 mg/kg compound of Formula (I) in 0.9% saline and the animals observed. Positive control mice are injected with Metrazole and 0.9% saline. Negative control mice are injected with 0.9% saline only. 3 mice are used in each control or experimental group. Mice are evaluated for seizure at 30 minutes, 90, 180, and 240 minutes after injection.

### Example 6. Effect of PPL-103 on Cocaine Self Administration

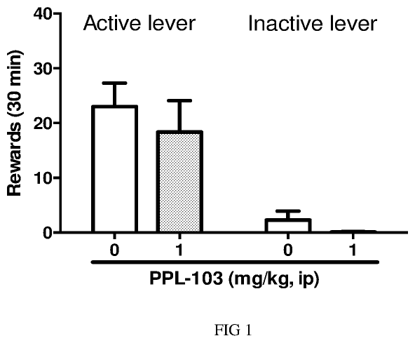

For self-administration experiments, male Sprague-Dawley rats (200-225 g) were used. Rats were housed in a room with a reverse 12-h light/12-h dark cycle. All self-administration experiments were conducted during the dark phase of the cycle. Under isoflurane anesthesia all rats were implanted with intravenous (iv) catheters into the right jugular vein to allow iv drug self-administration. Following recovery from surgery, animals were trained to self-administer cocaine (0.750 mg/kg/infusion in 1-hr daily sessions conducted in operant chambers equipped with two retractable levers. The number of lever presses were recorded. Cocaine infusions occurred by means of syringe pumps that were activated by responses on the ‘active’ lever, while responses on the other, ‘inactive’ lever controlled for non-specific motor behavior. Activation of the pump resulted in infusion of 0.1 ml of cocaine. Cocaine reinforcements were followed by a 20-sec time-out (TO) period (responses did not lead to programmed consequences) and accompanied by illumination of a cue light to signal delivery of cocaine. An intermittent tone (7 kHz, 70 dB) was sounded throughout sessions. Sessions were conducted under a FR-1 reinforcement schedule. N=7 animals were used in each of the PPL-103 and vehicle treated group. PPL-103 was found to produce a small but significant reduction in cocaine self administration (FIG. 1).

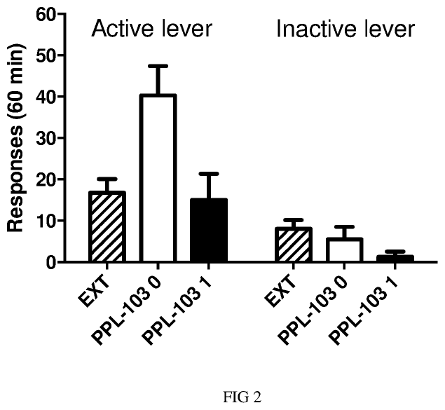

The effect of PPL-103 on reinstatement of cocaine seeking, a model of drug relapse, was also assessed. Cocaine-reinforced responding was extinguished in daily 1-hr sessions in which rats were free to press the lever in the absence of cocaine reward, with n=6 rats per group. This continued until ≤15 lever presses were achieved for three consecutive days. Extinction sessions (EXT) were conducted in the presence of cues (i.e., light, tone) and absence of cocaine. After the last EXT session, drug-prime-induced reinstatement of cocaine seeking was examined by injecting 10 mg/kg of cocaine (i.p.) immediately prior to a 1-hr reinstatement session that was carried out under conditions identical to that of EXT. To evaluate effects of PPL-103, 1 mg/kg of PPL-103, or vehicle was administered according to a counterbalanced Latin square design 15 min prior to the prime injection of cocaine. Responses on the previously cocaine-associated lever was recorded. Each reinstatement test was followed by two consecutive EXT sessions. PPL-103 completely blocked reinstatement of cocaine seeking (FIG. 2).

1. A method of treating pruritus, seizures, or a seizure disorder in a patient comprising administering a therapeutically effective amount of a compound of Formula (I), or pharmaceutically acceptable salt or hydrate thereof, to the patient, 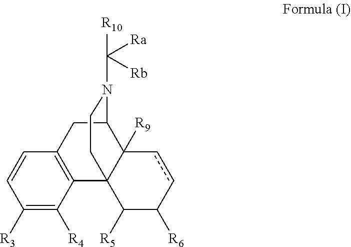
wherein, the dashed line in Formula (I) represents an optional double bond;
R₃ is hydrogen, halogen, hydroxyl, C₁-C₄alkyl, C₁-C₄alkoxy, or C₁-C₄alkyl ester;
R₄ and R₅ are both hydrogen or R₄ and R₅ are taken together to form a 5 membered ring, optionally containing one O or S ring atom;
R₆ is hydrogen, halogen, hydroxyl, oxo, C₁-C₄alkoxy, or C₁-C₄alkyl ester;
R₉ is hydrogen or methyl;
Rₐ and R_{b} are independently chosen from hydrogen, halogen, and C₁-C₄alkyl; and
R₁₀ is C₁-C₆alkyl, C₂-C₆alkenyl, or (C₃-C₆cycloalkyl)C₀-C₂alkyl.

2. The method of claim 1, wherein Rₐ and R_{b} are not both hydrogen.

3. The method of claim 1, wherein the compound or salt of Formula (I) is 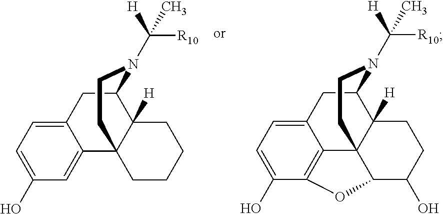
where R₁₀ is cyclopropyl, ethyl, propyl, or vinyl.

4-5. (canceled)

6. The method of claim 1, wherein the compound is 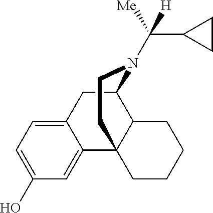
or a pharmaceutically acceptable salt thereof.

7. The method of claim 1, wherein the compound is 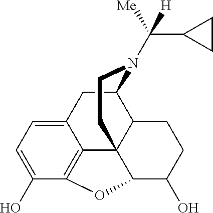
or a pharmaceutically acceptable salt thereof.

8. The method of claim 1, of treating pruritus comprising administering a sufficient amount of a compound or salt of Formula (I) to the patient so that the symptoms of pruritus are reduced compared to the symptoms prior to administration.

9. The method of claim 1, of treating pruritus comprising administering a sufficient amount of a compound or salt of Formula (I) to the patient so that the itch sensation of the patient is reduced compared to the patient's itch sensation prior to administration.

10. The method of claim 9, wherein the compound or salt of Formula (I) is administered orally.

11. (canceled)

12. The method of claim 1, wherein the compound of Formula (I) is administered together with an additional active agent effective for treating pruritus.

13-14. (canceled)

15. The method of claim 1, wherein the patient is suffering from a seizure or a seizure disorder.

16. The method of claim 15, wherein the patient is suffering from a seizure disorder and the amount of the compound of Formula I administered to the patient is an amount sufficient to decrease the frequency or severity of the patient's seizures compared to the frequency or severity of the patient seizures prior to administration of the compound of Formula (I).

17. The method of claim 15, wherein the patient is suffering from an acute seizure.

18-19. (canceled)

20. The method of claim 15, wherein the compound of Formula (I) is a first active agent and is administered in combination with an additional active agent.

21. The method of claim 20, wherein the additional active agent is an anti-convulsant or an anaesthetic.

22. The method of claim 20, wherein the additional active agent is an anti-convulsant and is administered chronically with the compound of Formula (I) in an amount sufficient to reduce seizure frequency or eliminate seizures in the patients.

23-27. (canceled)

28. A method of treating cocaine addiction in a cocaine addicted patient comprising administering a therapeutically effective amount of a compound of the formula 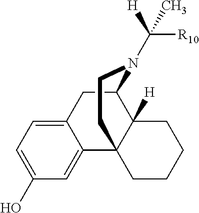
or a pharmaceutically acceptable salt thereof, wherein R₁₀ is cyclopropyl, ethyl, propyl, or vinyl.

29. The method of claim 28, wherein the compound is 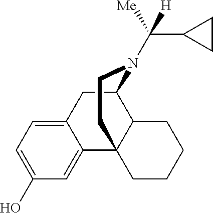
or a pharmaceutically acceptable salt thereof.

30. The method of claim 29, wherein the therapeutically effective amount is an amount sufficient for the patient to experience a reduced desire for cocaine compared to the patient's desire for cocaine prior to administration of the compound.

31. The method of claim 29, wherein the compound of salt of Formula (I) is administered orally.

32. The method of claim 29, wherein the compound is administered for a period of 1 to 10 weeks and the amount and frequency of dosage is such that concentration of the compound in the patient's plasma in never less than 50% of the patient's plasma Cmax.
